(19) United States
(12) Patent Application Publication
(10) Pub. No.: US 2019/0142737 A1
(43) Pub. Date: May 16, 2019

(54) SUBLINGUAL SPRAY FORMULATION COMPRISING DIHYDROARTEMESININ

(71) Applicant: LondonPharma Ltd., Norwich (GB)

(72) Inventor: CALVIN JOHN ROSS, Norfolk (GB)

(73) Assignee: LondonPharma Ltd., Norwich (GB)

(21) Appl. No.: 16/164,610

(22) Filed: Oct. 18, 2018

Related U.S. Application Data
(63) Continuation of application No. 15/697,240, filed on Sep. 6, 2017, now abandoned.
Application No. 15/697,240 is a continuation of application No. 14/813,424, filed on Jul. 30, 2015, now abandoned.
Application No. 14/813,424 is a continuation of application No. 13/265,518, filed on Dec. 1, 2011, now abandoned.

(30) Foreign Application Priority Data
Apr. 23, 2009 (GB) 0906971.7
Apr. 23, 2009 (GB) PCT/GB2009/050415

(51) Int. Cl. A61K 9/00; A61K 31/357; A61K 47/14; A61K 47/10

(52) U.S. Cl. CPC A61K 9/006; A61K 31/357; A61K 9/0043; A61K 47/10; A61K 47/14

(57) ABSTRACT

The invention provides pharmaceutical compositions for the treatment of neoplastic diseases, fluke infestations and Lyme disease, comprising compounds capable of providing dihydroartemesinin and a medium chain triglyceride formulated for transmucosal sublingual, buccal or nasal delivery, especially by a spray. Also provided are delivery devices containing the compositions.

### CROSS REFERENCE TO RELATED APPLICATIONS

This application is a continuation of U.S. application Ser. No. 15/697,240, filed Sep. 6, 2017, which is a continuation of U.S. application Ser. No. 14/813,424, filed Jul. 30, 2015, which is a continuation of U.S. application Ser. No. 13/265,518, filed Dec. 1, 2011, which is the National Stage of International Application No. PCT/GB2010/050672, filed Apr. 23, 2010, which is related to and claims the benefit of GB 0906971.7, filed Apr. 23, 2009 and PCT/GB2009/050415, filed Apr. 23, 2009. The entirety of each of these applications is hereby incorporated by reference for all purposes.

### FIELD OF THE INVENTION

The invention relates to pharmaceutical compositions, delivery methods, delivery devices and methods for the treatment of cancer. The invention also relates to pharmaceutical compositions, delivery methods, delivery devices and methods for the treatment of fluke infestations and Lyme disease (Borreliosis).

### BACKGROUND AND PRIOR ART KNOWN TO THE APPLICANT

Artemesinins, which may be isolated from the plant Artemesia annua are known for the treatment of malaria, and have also been shown to be effective for the treatment of a wide range of cancers, i.e. neoplasms, and especially malignant neoplasms. Amongst reported successes are the following:

Sing and Panwar (Integrative Cancer Therapies, 5(4): 2006, 391-394) report the treatment of pituitary adenoma with artemether.

Singh and Verma (Archive of Oncology, 10(4): 2002, 279-280) report the treatment of laryngeal squamous cell carcinoma with artesunate.

Singh and Lai (Life Sciences, 70(2001) 49-56) report the selective toxicity of dihydroartemesinin and holotransferrin toward human breast cancer cells.

Rowen (Townsend Letter for Doctors and Patients, December 2002) provides a summary of the use of artemisinins for the treatment of various cancers, including breast cancer, non-Hodgkin's Lymphoma, non-small cell lung carcinoma, and multiple skin cancers.

Efferth et al (“Anti-malaria drug is also active against cancer”, Int. J. Oncology, 18; 767-773, 2001) report activity of artemesinins against 55 cancer lines.

It is believed that the artemesinins have this broad effect on a large range of cancer cells because of their ability to react with ferrous iron to form free radicals: and most cancer cells have high rates of iron intake.

In addition, artemesinins have been shown to be effective in the treatment of liver flukes, and in particular schistosomiasis. Keiser and Morson (Exp. Parasitol., 118(2), 2008: 228-37) report the activity of artesunate and artemether against the liver fluke Fasciola hepatica.

Keiser et al (J. Antimicrobial Chemotherapy, 2006, 57, 1139-1145) also report that artesunate and artemether are effective fasciolicides.

Utzinger et al (Curr Opin Investig Drugs, 2007 Feb. 8(2), 105-16) report the use of artemesinins for treatment of individuals infested with Plasmodium spp. and Schistosoma haematobium with promising activity or artemesinins against intestinal and liver flukes, as well as against cancer cells.

Recent observations have also found that artemesinins are active against bacteria of the genus Borrelia, the causative agent of Lyme disease. Borrelia burgdorferi is the predominant cause of Lyme disease in the United States, Borrelia afzelii and Borrelia garinii being more common agents in most European cases.

Accordingly, amongst the active pharmaceuticals of use in the treatment of these conditions are a number of compounds derived from artemesenin, a sesquiterpene lactone endoperoxide originally isolated from Artemesia annua (Woodrow et al. Postgrad. Med. J. 2005; 81:71-78). These compounds include the semi-synthetic derivatives artenimol, artesunate, artemether and arteether (artemotil). The International Pharmacopoeia (Ph. Int., World Health Organisation) lists a number of these for the treatment of malaria (against which they are also active), viz: Artemether in the form of capsules, tablets or an injectable formulation; Artemesenin in the form of capsules or tablets; arteether in an injectable formulation; and both artenimol and artesunate in the form of tablets.

Once taken into the body, the artemesinins are converted to dihydroartemesinin and so these active compounds include all those that supply dihydroartemesinin in vivo.

One particular problem with the administration of artemesinins is their low bioavailability and the presence of a first pass effect when taken by the oral route, as will be discussed below. Furthermore, for long-term cancer treatment, it is particularly preferred that patients are able to either self-administer medication, or that medication can be administered by a non-qualified helper, and particularly in the home environment. This allows patients to remain at home, and reduces pressure on the healthcare system. Furthermore, cancer patients are often immune-compromised, and it is therefore particularly beneficial to keep them out of e.g. a hospital environment where the chances of contracting infections are higher. For these reasons at least, oral doses of artemesinins are not effective, especially for long-term treatment as might be required for cancer therapy, for treatment of fluke infestations or treatment of Lyme disease; injectable treatments are prone to risk of infection, need medically-qualified personnel and are not stable during storage; suppository administration is also not acceptable in many cultures, and might not be repeatably absorbed where patients are experiencing diarrhoea.

It can be seen that all of these formulations face the difficulties of administration described above. It is therefore amongst the objects of the present invention to address these and other issues.

### SUMMARY OF THE INVENTION

Accordingly, in a first aspect, the invention provides a pharmaceutical composition for the treatment of neoplasms comprising: a compound capable of providing dihydroartemesinin; and a pharmaceutically-acceptable excipient selected the group consisting of: medium chain length triglycerides; short chain triglycerides; omega-3-marine triglycerides; and fish oil, rich in omega-3-acids, said composition formulated for transmucosal sublingual, buccal or nasal dosage.

In a second aspect, the invention provides a pharmaceutical composition for the treatment of fluke infestation comprising: a compound capable of providing dihydroartemesinin; and a pharmaceutically-acceptable excipient selected the group consisting of: medium chain length triglycerides; short chain triglycerides; omega-3-marine triglycerides; and fish oil, rich in omega-3-acids, said composition formulated for transmucosal sublingual, buccal or nasal dosage.

In a third aspect, the invention provides a pharmaceutical composition for the treatment of Lyme disease (borreliosis) comprising: a compound capable of providing dihydroartemesinin; and a pharmaceutically-acceptable excipient selected the group consisting of: medium chain length triglycerides; short chain triglycerides; omega-3-marine triglycerides; and fish oil, rich in omega-3-acids, said composition formulated for transmucosal sublingual, buccal or nasal dosage.

The inventors have found that the transmucosal sub-lingual, transmucosal buccal and transmucosal nasal routes for administration of artemether or arteether are effective for delivery of the pharmaceutical into the systemic circulation e.g. for the treatment of cancer and fluke infestation. Furthermore, for the first time, it provides an administration route that is acceptable to patients requiring treatment, and that may be administered by non-medically qualified personnel. It has particular advantage, therefore, in treating these conditions. The composition can be delivered e.g. sublingually as a liquid bolus, or, more preferably, as a spray.

Medium chain length triglycerides are defined in the European Pharmacopoeia Monograph 0868, as:

A mixture of triglycerides of saturated fatty acids, mainly of caprylic acid (octanoic acid, C8H16O2) and of capric acid (decanoic acid, C10H20O2). Medium-chain triglycerides are obtained from the oil extracted from the hard, dried fraction of the endosperm of Cocos nucifera L. or from the dried endosperm of Elaeis guineensis Jacq. When Medium-chain Triglycerides are prepared from the endosperm of Cocos nucifera L., the title Fractionated Coconut Oil may be used. Medium chain length triglycerides have a minimum 95.0 percent of saturated fatty acids with 8 and 10 carbon atoms. Further chemical and physical properties are described in the European Pharmacopoeia Monograph 0868, and equivalent documents.

Short chain triglycerides are triglycerides having chain lengths of less than 6 carbon atoms.

Omega-3-marine triglycerides are defined in the European Pharmacopoeia Monograph 0868 as mixture of mono-, di- and triesters of omega-3 acids with glycerol containing mainly triesters and obtained either by esterification of concentrated and purified omega-3 acids with glycerol or by transesterification of the omega-3 acid ethyl esters with glycerol. The origin of the omega-3 acids is the body oil from fatty fish species coming from families like Engraulidae, Carangidae, Clupeidae, Osmeridae, Salmonidae and Scombridae. The omega-3 acids are identified as the following acids: alpha-linolenic acid (C18:3 n−3), moroctic acid (C18:4 n−3), eicosatetraenoic acid (C20:4 n−3), timnodonic (eicosapentaenoic) acid (C20:5 n−3; EPA), heneicosapentaenoic acid (C21:5 n−3), clupanodonic acid (C22:5 n−3) and cervonic (docosahexaenoic) acid (C22:6 n−3; DHA). The sum of the contents of the omega-3 acids EPA and DHA, expressed as triglycerides is a minimum of 45.0 percent, and the total omega-3 acids, expressed as triglycerides is a minimum of 60.0 percent. Tocopherol may be added as an antioxidant.

Fish oil, rich in omega-3-acids is also defined in the European Pharmacopeia as purified, winterised and deodorised fatty oil obtained from fish of the families Engraulidae, Carangidae, Clupeidae, Osmeridae, Scombridae and Ammodytidae. The omega-3 acids are defined as the following acids: alpha-linolenic acid (C18:3 n−3), moroctic acid (C18:4 n−3), eicosatetraenoic acid (C20:4 n−3), timnodonic (eicosapentaenoic) acid (C20:5 n−3; EPA), heneicosapentaenoic acid (C21:5 n−3), clupanodonic acid (C22:5 n−3) and cervonic (docosahexaenoic) acid (C22:6 n−3; DHA).

The content of the Fish oil, rich in omega-3-acids is as follows:

EPA, expressed as triglycerides: minimum 13.0 percent,

DHA, expressed as triglycerides: minimum 9.0 percent,

Total omega-3-acids, expressed as triglycerides: minimum 28.0 percent.

Authorized antioxidants in concentrations not exceeding the levels specified by the competent authorities may be added.

Whilst these definitions serve to define particularly preferred compositions of the recited excipients, the skilled addressee will appreciate that the composition of appropriate alternative excipients may also deviate from these exact compositional limits. Excipients of choice should exhibit analogous chemical properties such as the ability to solubilise artemether or arteether or other compounds providing dihydroartemesinin at the required concentration, not to degrade the pharmaceutically active ingredients, and to be non-toxic. The excipients should also have analogous physical properties such as at least being liquid at body temperature, and preferably having a suitable viscosity to allow the excipient to be used in preferred spray formulations described below. The viscosity for these applications should be low enough to be capable of atomizing, as described below, when used in a pump spray.

As an example, compositions might consist essentially of artemether or arteether and a pharmaceutically acceptable excipient consisting essentially of a triglyceride, liquid at 37° C., and medium chain triglycerides (as defined herein).

Particularly preferred compositions of the invention consist essentially of: artemether or arteether; and one or more pharmaceutically-acceptable excipients selected the group consisting of: medium chain length triglycerides; short chain triglycerides; and omega-3-marine triglycerides, said composition formulated for transmucosal sublingual, buccal or nasal dosage. The exclusion of significant amounts of other materials (e.g. higher molecular weight lipids) renders a composition that is ideally suited to transmucosal nasal, buccal, and especially sublingual delivery.

More preferred compositions comprise: artemether and a pharmaceutically-acceptable excipient selected the group consisting of: medium chain length triglycerides; short chain triglycerides; and omega-3-marine triglycerides, said composition formulated for transmucosal sublingual, buccal or nasal dosage, and especially a composition consisting essentially of: artemether and a pharmaceutically-acceptable excipient selected the group consisting of: medium chain length triglycerides; short chain triglycerides; and omega-3-marine triglycerides, said composition formulated for transmucosal sublingual, buccal or nasal dosage.

In any of these compositions, it is especially preferred that the composition is substantially free of water, as the inventors have found, contrary to accepted belief, that water can significantly reduce the shelf-life of the compositions, especially when stored at ambient temperatures. Preferred compositions would have less than 1% (w/w) water, and more preferably less than 0.5% (w/w) water, and most preferably less than 0.1% (w/w) water.

Also in any of these compositions, it is especially preferred that the composition is substantially free of ethanol. Again, the inventors have found that ethanol leads to degradation of the pharmaceutically active components. Preferred compositions in particular have less than 1% (w/w) ethanol, and more preferably less than 0.5% (w/w) ethanol and most preferably less than 0.1% (w/w) ethanol.

Also in any of these compositions, it is preferred that artemether or arteether is present at a concentration of between 2 and 250 milligrams per gram of excipient. This concentration provides an appropriate level for the expected volumes used for the described transmucosal delivery. More preferably, the composition comprises: artemether or arteether, dissolved in the excipient at a concentration of between 2 and 200 milligrams per gram of excipient. Other preferred concentrations are between 2 and 100 milligrams per gram; between 2 and 50 milligrams per gram. The lower concentrations provide compositions particularly suitable for paediatric use, and are also more likely to ensure that the pharmaceutically active components remain in solution over a wide temperature range, rather than having some portion as e.g. a suspension. This is particularly important to ensure that delivery of the drug is by the recited transmucosal route. If significant amounts of the active components are not in solution, then there is an increased likelihood that some will be swallowed, thereby reducing the beneficial effects of such transmucosal delivery described below.

In especially preferred compositions, the said excipient comprises a medium chain triglyceride, said triglyceride comprising a minimum of 95 percent of saturated fatty acids with between 6 and 12 carbon atoms. More preferably, said excipient comprises a medium chain triglyceride, said triglyceride comprising a minimum of 95 percent of saturated fatty acids with between 8 and 10 carbon atoms.

Also in any such composition, it is also particularly preferred that the composition further comprises an essential oil such as menthol, vanillin or orange oil, lemon oil, clove oil, peppermint oil, spearmint oil. Particular technical advantages of such an essential oil, especially menthol, which acts as a solubilising agent, are described further below. In addition to any solubilising effect such essential oils also act as flavourings, having a number of benefits: the flavours mask unpleasant tastes of the medicament thereby leading to increased patient compliance. This is particularly important for such essentially liquid-based formulations which cannot by their nature be encapsulated or “sugar-coated”. The flavours also give a feedback to the user or administrator of the medication that the medication has been successfully delivered (the patient can taste it), and furthermore that it has been delivered to the correct place.

In a second aspect, the invention provides a medicament delivery device containing a composition described herein, said device adapted to deliver individual or successive doses of said composition, each individual or successive dose having a volume of less than 1000 microlitres. The use of small dose volumes reduces the likelihood that the composition will be swallowed, or spat out, by the patient. The likelihood is reduced further by use of smaller volumes (especially in the paediatric context or for nasal delivery) and so in further preferred embodiments, each successive dose has a volume of less than 600 microlitres; less than 400 microlitres; less than 200 microlitres; or even less than 100 microlitres. Smaller volumes are especially preferred for paediatric use, or nasal delivery.

In a third aspect, the invention provides a medicament delivery device containing a composition described herein, said device and composition adapted to deliver individual or successive doses of said composition, each individual or successive dose containing no more than 100 mg, and preferably no more than 80 mg of a compound capable of providing dihydroartemesinin, such as artemether or arteether. Such devices are preferably adapted to assist sublingual delivery, especially by non-medically trained personnel. Limiting the amount of active pharmaceutical delivered with each dose is especially important in the context of treatment by less skilled personnel (e.g. self-administration by a patient in a domestic setting, which is likely for long-term anti cancer therapy) to ensure that over-dosing is avoided. Preferably, said device and composition adapted to deliver individual or successive doses of said composition, each individual or successive dose containing no more than 10 mg of a compound capable of providing dihydroartemesinin, such as artemether or arteether. This provides an appropriate device for paediatric use.

Preferably, the delivery devices according to these aspects comprise a spray, and especially a pump spray. The use of a pump spray increases the area of mucosa to which the composition is applied, thereby increasing absorption and minimising the likelihood that the medicament is swallowed. More preferably, said device is adapted to produce a spray of composition having a mean droplet diameter greater than 20 microns, or even greater than 50 microns, or preferably greater than 75 microns. In this way, inadvertent delivery of the medicament to the lungs is avoided, or reduced.

In a fourth aspect, the invention also provides a device for providing pharmaceutical doses comprising a container containing a pharmaceutical composition described herein, and valve means arranged to transfer doses of said pharmaceutical composition to the exterior of the container. Such a device may be attached to e.g. a separate transmucosal buccal, nasal or sublingual delivery device, such as a spray.

In a fifth aspect, the invention provides a kit for the treatment of neoplasms, fluke infestation or Lyme disease comprising a composition described herein and instructions to administer said composition to a patient in need thereof by the transmucosal sublingual, buccal or nasal route. Preferably, said kit has instructions to administer said composition to a patient in need thereof by the sublingual route.

In a sixth aspect, the invention provides a method of treating neoplastic diseases, fluke infestation or Lyme disease comprising the administration to a patient in need thereof of a therapeutically effective amount of a compound providing dihydroartesinin (e.g. an artemesinin, and especially artemether or arteether) by the transmucosal sublingual, buccal or nasal route. More preferably, said administration is by the sublingual route.

Also included in the scope of the invention is the use of a compound providing dihydrartemesinin in the preparation of a pharmaceutical composition according to any of the aspects, or preferred aspects, described above for the treatment of neoplastic diseases, fluke infestation or Lyme disease.

Preferably, any of the pharmaceutical compositions or devices provided by the present invention are for the treatment of neoplasms, fluke infestation or Lyme disease.

In any of the compositions of the invention it is particularly preferred that the composition also includes a transferrin, such as holotransferrin, as this enhances the action of dihydroartemesinin.

In any methods of treatment of the invention it is also particularly preferred to co-administer a transferrin, such as holotransferrin, as this enhances the action of dihydroartemesinin.

In any methods of treatment of the invention it is also particularly preferred that said compound providing dihydroartemesinin is formulated in a composition described above.

Also included within the scope of the invention are pharmaceutical compositions, medicament delivery devices, kits and methods substantially as described herein, with reference to, and as illustrated by any appropriate combination of the accompanying drawings.

### DISCLAIMED EMBODIMENTS

In preferred embodiments of the invention, the following numbered aspects, disclosed in co-pending International Patent Application PCT/GB2008/050999 are particularly disclaimed:

1. A pharmaceutical composition comprising:

artemether or arteether; and
a pharmaceutically-acceptable excipient selected the group consisting of:
medium chain length triglycerides;
short chain triglycerides;
omega-3-marine triglycerides; and
fish oil, rich in omega-3-acids
said composition formulated for transmucosal sublingual, buccal or nasal dosage.

2. A composition according to aspect 1 consisting essentially of:

artemether or arteether; and
one or more pharmaceutically-acceptable excipients selected the group consisting of:
medium chain length triglycerides;
short chain triglycerides;
omega-3-marine triglycerides; and
fish oil, rich in omega-3-acids
said composition formulated for transmucosal sublingual, buccal or nasal dosage.

3. A composition according to aspect 1 comprising:

artemether and
a pharmaceutically-acceptable excipient selected the group consisting of:
medium chain length triglycerides;
short chain triglycerides;
omega-3-marine triglycerides; and
fish oil, rich in omega-3-acids
said composition formulated for transmucosal sublingual, buccal or nasal dosage.
4. A composition according to aspect 1 consisting essentially of:
artemether and
one or more pharmaceutically-acceptable excipients selected the group consisting of:
medium chain length triglycerides;
short chain triglycerides; and
omega-3-marine triglycerides; and
fish oil, rich in omega-3-acids
said composition formulated for transmucosal sublingual, buccal or nasal dosage.

5. A composition according to aspect 1 consisting essentially of:

artemether or arteether; and
a pharmaceutically acceptable excipient consisting essentially of:
a triglyceride, liquid at 37° C.; and
medium chain length triglycerides;
said composition formulated for transmucosal sublingual, buccal or nasal dosage.

6. A composition according to any preceding aspect, substantially free of water.

7. A composition according to any preceding aspect, substantially free of ethanol.

8. A pharmaceutical composition according to any preceding aspect wherein artemether or arteether is present at a concentration of between 2 and 250 milligrams per gram of excipient.

9. A composition according to any preceding aspect wherein said excipient comprises a medium chain triglyceride, said triglyceride comprising a minimum of 95 percent of saturated fatty acids with between 6 and 12 carbon atoms.

10. A composition according to aspect 8 wherein said excipient comprises a medium chain triglyceride, said triglyceride comprising a minimum of 95 percent of saturated fatty acids with between 8 and 10 carbon atoms.

11. A composition according to any preceding aspect further comprising an essential oil such as menthol, vanillin or orange oil, lemon oil, clove oil, peppermint oil, spearmint oil.

12. A composition according to any preceding aspect for the treatment or prophylaxis of malaria.

13. A composition according to any preceding aspect formulated for sublingual delivery.

14. A medicament delivery device containing a composition according to any preceding aspect, said device adapted to deliver individual or successive doses of said composition, each individual or successive dose having a volume of less than 1000 microlitres.

15. A medicament delivery device containing a composition according to any preceding aspect, said device and composition adapted to deliver individual or successive doses of said composition, each individual or successive dose containing no more than 80 mg of artemether or arteether.

16. A medicament delivery device containing a composition according to any preceding aspect, said device and composition adapted to deliver individual or successive doses of said composition, each individual or successive dose containing no more than 10 mg of artemether or arteether.

17. A delivery device according to any of aspects 14 to 16 wherein said device comprises a pump spray.

18. A delivery device according to aspect 17 wherein said device is adapted to produce a spray of composition having a mean droplet diameter greater than 20 microns.

19. A device for providing pharmaceutical doses comprising a container containing a pharmaceutical composition according to any of aspects 1 to 13, and valve means arranged to transfer doses of said pharmaceutical composition to the exterior of the container.

20. A kit for the treatment or prophylaxis of malaria comprising a composition according to any of aspects 1 to 13 and instructions to administer said composition to a patient in need thereof by the transmucosal sublingual, buccal or nasal route.

21. A kit for the treatment or prophylaxis of malaria comprising a composition according to any of aspects 1 to 13 and instructions to administer said composition to a patient in need thereof by the sublingual route.

22. A method of treating a disease responsive to artemether or arteether comprising the administration to a patient in need thereof of a therapeutically effective amount of artemether or arteether by the transmucosal sublingual, buccal or nasal route.

23. A method of treating a disease responsive to artemether comprising the administration to a patient in need thereof of a therapeutically effective amount of artemether by the transmucosal sublingual, buccal or nasal route.

24. A method according to either aspect 22 or 23 wherein said administration is by the sublingual route.

25. A method according to any of aspects 22 to 24 wherein said disease is malaria.

26. A kit for the treatment of malaria comprising a composition according to any of aspects 1 to 13 and instructions to administer said composition to a patient in need thereof by the transmucosal sublingual, buccal or nasal route.

27. A kit for the treatment of malaria comprising a composition according to any of aspects 1 to 13 and instructions to administer said composition to a patient in need thereof by the transmucosal sublingual route.

### DESCRIPTION AND PREFERRED EMBODIMENTS OF THE INVENTION

One of the most important aspects of providing a clinically useful treatment for diseases, infections or infestations responsive to dihydroartemesinin (produced in vivo by metabolisms of an artemesinin such as artemether, arteether and artesunate) is to provide a formulation and an administration route for the active ingredient that can withstand the challenges of those communities where the disease is an especially acute problem. For example, any formulation needs to be stable for long periods of time, and at the relatively high temperatures encountered in countries where e.g. schistosomiasis is endemic. The medicament will often need to be administered (without delay) to individuals who are weak, perhaps malnourished, and possibly suffering from vomiting and diarrhoea. In many cases, the medicament may also need to be administered by non-medically-trained personnel. It is also important for any active ingredient to have good (and consistent) bioavailability, to ensure that the drug reaches the site of action without adverse side effects.

In order to address these problems, the inventors have found that the transmucosal sublingual, buccal or nasal route of administration of artemether provides a greater likelihood of higher and more reproducible levels of bioavailability than that demonstrated by the oral (i.e. swallowed) or intramuscular route. Navaratnam et al (Clin Pharmacokinet, 2000, October; 39(4): 255-270) report the bioavailability of artemether in animals by oral administration to be as low as 19-35%, and only 54% when administered by intramuscular injection. In humans, the bioavailability of artemether was low in both the intramuscular (25%) and intrarectal (35%) route, with considerable variability in absorption. The authors report that “Preliminary studies in children with cerebral malaria indicated that the bioavailability of intramuscular artemether is highly variable and could potentially affect treatment outcome in the most severely ill patients”.

The use of the transmucosal sublingual, buccal or nasal route of administration avoids the first-pass effect that occurs with oral and rectal administration. Whilst adults might be able to tolerate the large oral doses of artemether required to overcome the low bioavailability of the drug for short periods of time, this is not the case in children, and so the compositions disclosed herein are particularly suitable for the treatment of diseases such as cancer that might require protracted periods of medication, or that might be for paediatric use.

Preliminary results of initial, confidential, dose ranging studies are presented below, indicating surprisingly increased bioavailability of the drug when administered by sublingual spray in comparison to oral administration by tablet.

The inventors have also found that, contrary to accepted belief, artemether is not stable when in contact with water, ethanol, or propellants that might be used for aerosol formulations.

Tables 1 and 2 show impurities present in Artemether API, and artemether in three solvent systems: 20% ethanol+80% propellant; 50% ethanol+50% propellant; 100% ethanol; and a medium chain triglyceride, in this case, the triglyceride sold under the registered trade mark Miglyol® 810. Miglyol® is a medium chain triglyceride containing saturated C8 and C10 fatty acids, typically between 65-80% of caprylic acid (C8:0) and 20-35% of capric acid (C10:0).

The propellant used in these test was 1,1,1,2 tetrafluoroethane, sold under the registered trade mark Zephex® 134a. Similar results were obtained for the propellants butane, Zephex® 227 (1,1,1,2,3,3,3 heptafluoropropane) and for a mixture of butane and propane.

Table 1 shows the impurities (as a percentage of the peak area of an HPLC chromatogram of artemether) after storage of the compositions at 30° C. for eight weeks. Table 2 shows the corresponding impurities after storage for eight weeks at 40° C.

TABLE 1
Storage at 30° C.

| | Relative Retention Time: | | | | | |
|---|---|---|---|---|---|---|
| | 0.35 | 0.68 | 0.73 | 0.87 | 0.91 | 1.17 |
| | % of artemether | | | | | |
| Artemether API | 0.4 | | 0.1 | 0.2 | | |
| 20% EtOH 80% propellant | 1.6 | 0.3 | 0.7 | 0.2 | 1.3 | 0.2 |
| 50% EtOH 50% propellant | 1.0 | 0.2 | 0.5 | 0.2 | 1.5 | 0.2 |
| 100% EtOH | 0.3 | 0.2 | 0.5 | 0.2 | | |
| Miglyol 810 ® | 0.4 | | 0.1 | 0.2 | | |

TABLE 2
Storage at 40° C.

| | Relative Retention Time: | | | | | |
|---|---|---|---|---|---|---|
| | 0.35 | 0.68 | 0.73 | 0.87 | 0.91 | 1.17 |
| | % of artemether | | | | | |
| Artemether API | 0.4 | | 0.1 | 0.2 | | |
| 20% EtOH 80% propellant | 4.9 | 1.9 | 2.9 | 0.2 | 5.3 | 1.4 |
| 50% EtOH 50% propellant | 2.2 | 1.4 | 2.5 | 0.2 | 4.8 | 1.0 |
| 100% EtOH | 2.2 | 0.7 | 1.6 | 0.2 | 1.0 | 0.7 |
| Miglyol 810 ® | 0.6 | | 0.1 | 0.2 | | |

Representative chromatograms are shown in FIG. 13. It can be seen that the levels of impurities in the Miglyol® 810 formulation are not significantly higher than those observed in the initial Artemether API. In all other cases, the impurities are at levels that exceed those permitted under the ICH Harmonised Tripartite Guidelines for Impurities in New Drug Products without specific identification or further toxicological examination.

A solution in a medium chain triglyceride, especially a saturated triglyceride such as Miglyol® 810 therefore constitutes a stable formulation for the active ingredient. Being a saturated triglyceride, it is believed that this confers stability to the artemether. Given its chemical structure, it is likely that the main route of degradation of artemether is via reduction mechanisms, which might explain the protection afforded by such saturated fatty acid-containing triglycerides.

When used in a spray delivery system, e.g. in a manually-actuated pump spray, the triglyceride also acts as a pump and valve lubricant, thereby removing the need to add additional lubricants to the formulation. The use of such medium chain triglycerides also produces a formulation of appropriate viscosity and surface tension for use in a pump spray delivery system.

Further advantages also flow from the use of medium chain triglyceride: being hydrophobic, the triglyceride adheres to the mucosa of the mouth, and so allows time for the artemether to be absorbed transmucosally. The hydrophobic nature of the composition resists being washed out of the mouth by the action of saliva, which would otherwise cause the active ingredient to be swallowed.

In especially preferred embodiments of the invention, the artemether-triglyceride solution is supplemented with menthol, or alternatively with orange oil or vanilla. The inventors have found that this has a number of benefits:

(1) Its function as a taste-masking agent is particularly important in the context of administration of drugs to children or to patients who need to take the medication over prolonged periods of time; any bad taste of the drug experienced by the patient makes patient compliance less likely.

(2) The essential oil also acts as a penetration enhancer to improve the uptake of the pharmaceutical ingredient through the mucosa of the mouth.

(3) The addition of a flavour also allows the person administering the drug to check firstly that the drug has been dispensed (the patient can taste or smell it) and secondly that it has been dispensed into the right place—if the drug were e.g. accidentally dispensed directly into the throat, there would be no taste sensation.

(4) A surprising feature is that the essential oil (especially levomenthol) also assists with the solubilisation of the artemether. In a solubility trial, dissolution of artemether in miglyol occurred after 4 minutes 30 seconds when menthol added before artemether compared to 5 minutes 55 seconds when artemether added before menthol.

As an example, preferred formulations (for sublingual or buccal paediatric use) are given in Tables 3 and 4. For adult use, or for the treatment of some indications, concentrations higher or lower than those exemplified are envisaged. Two different dose concentrations are given suitable for use in a spray delivery system. A number of sprays (i.e. individual spray actuations of 100 microlitres) may be given, dependent on the weight of the child to be treated:

TABLE 3
3 mg Artemether per actuation

| Raw Material Item | Weight (g) | % w/w |
|---|---|---|
| Artemether IP | 0.090 | 3.2 |
| Levomenthol Ph. Eur. | 0.020 | 0.7 |
| Miglyol ® 810 | 2.690 | 96.1 |

TABLE 4
6 mg Artemether per actuation

| Raw Material Item | Weight (g) | % w/w |
|---|---|---|
| Artemether IP | 0.180 | 6.4 |
| Levomenthol Ph. Eur. | 0.020 | 0.7 |
| Miglyol ® 810 | 2.600 | 92.9 |

Table 5 outlines an example of a preferred dosage regime for paediatric use. Alternative regimes are envisaged, e.g. dosing at 3 mg/kg body weight.

TABLE 5
Paediatric Dosage Regime

| | Number of | | Number of | Total |
|---|---|---|---|---|
| | doses at 3 mg | | doses at 6 mg | delivered |
| Weight of | Dose per spray | Total delivered | Dose per spray | dose |
| child (kg) | actuation | dose mg/kg | actuation | mg/kg |
| 3 | 1 | 1.00 | | |
| 4 | 1 | 0.75 | | |
| 5 | 2 | 1.20 | | |
| 6 | 2 | 1.00 | | |
| 7 | 2 | 0.86 | | |
| 8 | 3 | 1.13 | | |
| 9 | 3 | 1.00 | | |
| 10 | 3 | 0.90 | | |
| 11 | 4 | 1.09 | | |
| 12 | 4 | 1.00 | 2 | 1.00 |
| 13 | 4 | 0.92 | 2 | 0.92 |
| 14 | 5 | 1.07 | 2 | 0.86 |
| 15 | 5 | 1.00 | 3 | 1.20 |
| 16 | 5 | 0.94 | 3 | 1.13 |
| 17 | | | 3 | 1.06 |
| 18 | | | 3 | 1.00 |
| 19 | | | 3 | 0.95 |
| 20 | | | 3 | 0.90 |
| 21 | | | 3 | 0.86 |
| 22 | | | 4 | 1.09 |
| 23 | | | 4 | 1.04 |
| 24 | | | 4 | 1.00 |
| 25 | | | 4 | 0.96 |
| 26 | | | 4 | 0.92 |
| 27 | | | 4 | 0.89 |
| 28 | | | 5 | 1.07 |
| 29 | | | 5 | 1.03 |
| 30 | | | 5 | 1.00 |

Formulations for adult use may be prepared at higher concentrations of artemether, such as 150-200 mg/ml. For adult use, individual spray volumes may be larger than the 100 microlitre example described here for paediatric use.

Bioavailability of Artemether

The applicant has carried out confidential trials to assess the uptake of the artemether-containing compositions of the present invention when delivered by the sublingual route, by comparison to oral administration by tablet.

Trials were carried out on healthy male adult human volunteers (16 subjects per cohort), and subject to normal ethical approval. Three single-dose regimes according to the present invention were studied, and compared to a regime using oral-dosed tablets, as follows:

Sub-Lingual Spray Regimes

Spray formulations of artemether were prepared as detailed above, and administered, on a single occasion, to a group of volunteers by the sublingual route. A number of successive actuations of the spray were administered, as shown in Table 6, below.

TABLE 6
Dosage Regime for Single Dose Study
Sublingual Spray Formulation

| | | Dose per | Number of | |
|---|---|---|---|---|
| Test | Formulation | Actuation (mg) | Actuations | Total Dose (mg) |
| T1 | As Table 3 | 3 | 5 | 15 |
| T2 | As Table 3 | 3 | 10 | 30 |
| T3 | As Table 4 | 6 | 5 | 30 |

Reference Oral Dose

As a reference, a fourth group of volunteers were administered tablets containing artemether, on a single occasion, as shown in Table 7, below.

TABLE 7
Dosage Regime for Single Dose Study
Oral Tablet Formulation

| | | Dose per Tablet | Number of | |
|---|---|---|---|---|
| Test | Formulation | (mg) | Tablets | Total Doge (mg) |
| T4 | Tablet | 10 | 3 | 30 |

Following administration of each dosage regime, blood samples were taken from the subjects, and plasma concentrations of artemether and its immediate metabolite dihydroartemesinin were determined, in order to compare bioavailability by the two routes.

FIGS. 1-6 show mean plasma concentration of artemether following two comparison dose regimes. FIGS. 7-12 show the corresponding mean plasma concentration of dihydroartemesinin.

FIGS. 1 and 7 compare regimes T1 (open squares) and T4 (closed circles): 15 mg artemether via 5 sublingual spray doses vs. 30 mg artemether via tablet.

FIGS. 2 and 8 compare regimes T2 (open squares) and T4 (closed circles): 30 mg artemether via 10 sublingual spray doses vs. 30 mg artemether via tablet.

FIGS. 3 and 9 compare regimes T3 (open squares) and T4 (closed circles): 30 mg artemether via 5 sublingual spray doses vs. 30 mg artemether via tablet.

FIGS. 4 and 10 compare regimes T1 (open squares) and T2 (closed circles): 15 mg artemether via 5 sublingual spray doses vs. 30 mg artemether via 10 sublingual spray doses.

FIGS. 5 and 11 compare regimes T2 (open squares) and T3 (closed circles): 30 mg artemether via 10 sublingual spray doses vs. 30 mg artemether via 5 sublingual spray doses.

FIGS. 6 and 12 compare regimes T1 (open squares) and T3 (closed circles): 15 mg artemether via 5 sublingual spray doses vs. 30 mg artemether via 5 sublingual spray doses).

Pharmacokinetic data for each of the four dosage regimes are given in Tables 8-11, below:

TABLE 8
Test Group T1
Single sublingual administration of 15 mg Artemether sublingual spray:
3 mg per actuation

| | Plasma Artemether | Plasma Dihydroartemesinin |
|---|---|---|
| Pharmacokinetic | (n = 16) | (n = 16) |
| Parameters* | (mean ± SD) | (mean ± SD) |
| AUC₀₋₁₂ (ng · h/mL) | 25.85 ± 13.88 | 29.63 ± 11.58 |
| Cₘₐₓ (ng/mL) | 16.11 ± 8.69 | 18.29 ± 7.52 |
| Tₘₐₓ (h) | 1.70 ± 0.68 | 1.83 ± 0.68 |
| t_{1/2} (h) | 0.72 ± 0.30 | |
| λ_{z} (h⁻¹) | 1.11 ± 0.40 | |
| CL/F (ng/h) | 0.74 ± 0.46 | 0.54 ± 0.15 |
| V/F (L) | 0.68 ± 0.33 | 0.51 ± 0.16 |

*Key:
AUC₀₋₁₂ (ng · h/mL) Area under the concentration curve between 0-12 h.
Cₘₐₓ (ng/mL) Maximum observed plasma concentration
Tₘₐₓ (h) Time of observed maximum plasma concentration
t_{1/2} (h) Elimination half-life
λ_{z} (h⁻¹) Elimination rate constant
CL/F (ng/h) Apparent clearance rate
V/F (L) Apparent volume of distribution

TABLE 9
Test Group T2
Single sublingual administration of 30 mg Artemether sublingual spray:
3 mg per actuation

| | Plasma Artemether | Plasma Dihydroartemesinin |
|---|---|---|
| Pharmacokinetic | (n = 16) | (n = 16) |
| Parameters | (mean ± SD) | (mean ± SD) |
| AUC₀₋₁₂ (ng · h/mL) | 76.60 ± 43.12 | 99.51 ± 50.33 |
| Cₘₐₓ (ng/mL) | 32.12 ± 16.39 | 44.11 ± 28.48 |
| Tₘₐₓ (h) | 1.73 ± 0.82 | 2.10 ± 1.17 |
| t_{1/2} (h) | 1.39 ± 0.49 | |
| λ_{z} (h⁻¹) | 0.56 ± 0.20 | |
| CL/F (ng/h) | 0.56 ± 0.37 | 0.36 ± 0.13 |
| V/F (L) | 1.00 ± 0.55 | 0.72 ± 0.36 |

Key as Table 8

TABLE 10
Test Group T3
Single sublingual administration of 30 mg Artemether sublingual spray:
6 mg per actuation

| | Plasma Artemether | Plasma Dihydroartemesinin |
|---|---|---|
| Pharmacokinetic | (n = 16) | (n = 16) |
| Parameters | (mean ± SD) | (mean ± SD) |
| AUC₀₋₁₂ (ng · h/mL) | 71.11 ± 41.08 | 86.19 ± 27.68 |
| Cₘₐₓ (ng/mL) | 35.24 ± 23.91 | 41.14 ± 16.45 |
| Tₘₐₓ (h) | 1.67 ± 0.77 | 1.88 ± 0.74 |
| t_{1/2} (h) | 1.40 ± 0.59 | |
| λ_{z} (h⁻¹) | 0.59 ± 0.25 | |
| CL/F (ng/h) | 0.63 ± 0.49 | 0.39 ± 0.15 |
| V/F (L) | 1.01 ± 0.49 | 0.91 ± 0.67 |

Key as Table 8

TABLE 11
Test Group T4
Single oral administration of 30 mg Artemether Tablets
10 mg per Tablet

| | Plasma Artemether | Plasma Dihydroartemesinin |
|---|---|---|
| Pharmacokinetic | (n = 16) | (n = 16) |
| Parameters | (mean ± SD) | (mean ± SD) |
| AUC₀₋₁₂ (ng · h/mL) | 34.59 ± 21.01 | 38.49 ± 12.38 |
| Cₘₐₓ (ng/mL) | 10.12 ± 7.19 | 10.99 ± 4.39 |
| Tₘₐₓ (h) | 1.02 ± 0.86 | 1.39 ± 0.88 |
| t_{1/2} (h) | 3.44 ± 4.26 | |
| λ_{z} (h⁻¹) | 0.31 ± 0.15 | |
| CL/F (ng/h) | 1.11 ± 1.01 | 0.76 ± 0.23 |
| V/F (L) | 3.90 ± 2.90 | 2.36 ± 1.26 |

Key as Table 8

From these preliminary results, it can be seen that comparison of the area under the plasma concentration curve during the 12 hours following the doses (AUCO-12), a well-accepted measure of absorption, shows significant and surprisingly higher absorption of artemether when administered sublingually as a spray formulation as disclosed herein by comparison to oral tablet dosing.

For comparison of bioavailability of artemether via the sublingual spray route described herein with administration by oral tablets, we have calculated the F-values, commonly used to compare two dose regimes, generally A and B, for the artemether data, as follows:

$${F}_{A \text{-} B} = \frac{{AUC}_{A}}{{AUC}_{B}} \frac{{dose}_{B}}{{dose}_{A}}$$

The results are as follows:

F_{T1-T4}=1.67±0.60 (S.D.)

F_{T2-T4}=2.24±0.92 (S.D.)

F_{T3-T4}=2.09±0.69 (S.D.)

This indicates that approximately between 1.7 and 2.2 times more artemether was absorbed when administered as a sublingual spray as described herein by comparison to oral administration by tablet, despite the oral dose being twice as large in the first instance. The indicative bioavailability by the sublingual route is therefore at least twice that by the oral route for equivalent doses.

Inspection of the data of Tables 8-11, and FIGS. 1-12 also confirms this general finding for the primary active metabolite of artemether (dihydroartemesinin).

Avoidance of Autoinduction

It is known that both oral and rectal administration of artemesinins is associated with autoinduction of the drug metabolism in individuals (see e.g. Ashton M, Hai T N, Sy N D, Huong D X, Van Huong N, Nieu N T, Cong L D. “Artemisinin pharmacokinetics is time-dependent during repeated oral administration in healthy male adults.”, Drug Metab Dispos. 1998; 26:25-7, and “Retrospective analysis of artemisinin pharmacokinetics: application of a semiphysiological autoinduction model”, Asimus and Gordi, Br. J Clin Pharmacol. 2007 June; 63(6): 758-762). As a result, systemically circulating artemesinin declines with each successive dose, thereby reducing the effectiveness of drug dosage regimes.

In confidential trials, the inventors have found that administration of artemesinins by the transmucosal sublingual route avoids such autoinduction, leading to consistent uptake and accumulating systemic concentration of the active drug metabolite, dihydroartemesinin, thereby providing significant advantage in administration by the sublingual route. A similar avoidance of autoinduction is expected with delivery by the transmucosal buccal or nasal route.

In confidential trials, volunteers followed the following treatment: A single administration of 30 mg artemether sublingual spray 6 mg/actuation on days 1 and 5 following an overnight fast, and twice daily administrations of 30 mg artemether sublingual spray 3 mg/actuation on days 2, 3, and 4 following a morning or evening meal. Blood samples were collected for pharmacokinetic analysis at the following time points:

Day 1: Predose, 0.25, 0.5, 0.75, 1, 1.5, 2, 2.5, 3, 4, 6, 8, and 12 h after dosing.

Days 2, 3, and 4: pre morning dose and 0.5, 1, 2 and 4 h after morning dose and pre evening dose and 1 hour after evening dose.

Day 5: Predose, 0.25, 0.5, 0.75, 1, 1.5, 2, 2.5, 3, 4, 6, 8, 12 h and 24 h after dosing.

Pharmacokinetic analysis of plasma dihydroartemesinin on days 1 and 5 revealed an effectively identical response, indicating the lack of autoinduction. Plasma concentration curves are shown in FIG. 14.

### FIGURE CAPTIONS

FIG. 1: Plot of mean plasma Artemether concentration vs time with standard deviation following a single sublingual administration of 15 mg Artemether Sublingual Spray 3 mg/actuation (T1) and single oral administration of 30 mg Artemether Tablets 10 mg/tablet (T4). Mean±SD (⋅=reference, T4, □=test, T1)

FIG. 2: Plot of mean plasma Artemether concentration vs time with standard deviation following a single sublingual administration of 30 mg Artemether Sublingual Spray 3 mg/actuation (T2) and single oral administration of 30 mg Artemether Tablets 10 mg/tablet (T4). Mean±SD (⋅=reference, T4, □=test, T2)

FIG. 3: Plot of mean plasma Artemether concentration vs time with standard deviation following a single sublingual administration of 30 mg Artemether Sublingual Spray 6 mg/actuation (T3) versus single oral administration of 30 mg Artemether Tablets 10 mg/tablet (T4). Mean±SD (⋅=reference, T4, □=test, T3)

FIG. 4: Plot of mean plasma artemether concentration vs time with standard deviation following a single sublingual administration of 15 mg Artemether Sublingual Spray 3 mg/actuation (T1) versus single sublingual administration of 30 mg Artemether Sublingual Spray 3 mg/actuation (T2). Mean±SD (⋅=reference, T2, □=test, T1)

FIG. 5: Plot of mean plasma Artemether concentration vs time with standard deviation following a single sublingual administration of 30 mg Artemether Sublingual Spray 3 mg/actuation (T2) versus single sublingual administration of 30 mg Artemether Sublingual Spray 6 mg/actuation (T3). Mean±SD (⋅=reference, T3, □=test, T2)

FIG. 6: Plot of mean plasma Artemether concentration vs time with standard deviation following a single sublingual administration of 15 mg Artemether Sublingual Spray 3 mg/actuation (T1) versus single sublingual administration of 30 mg Artemether Sublingual Spray 6 mg/actuation (T3). Mean±SD (⋅=reference, T3, □=test, T1)

FIG. 7: Plot of mean plasma Dihydroartemisinin concentration vs time with standard deviation following a single sublingual administration of 15 mg Artemether Sublingual Spray 3 mg/actuation (T1) and single oral administration of 30 mg Artemether Tablets 10 mg/tablet (T4). Mean±SD (⋅=reference, T4, □=test, T1)

FIG. 8: Plot of mean plasma Dihydroartemisinin concentration vs time with standard deviation following a single sublingual administration of 30 mg Artemether Sublingual Spray 3 mg/actuation (T2) and single oral administration of 30 mg Artemether Tablets 10 mg/tablet (T4). Mean±SD (⋅=reference, T4, □=test, T2)

FIG. 9: Plot of mean plasma Dihydroartemisinin concentration vs time with standard deviation following a single sublingual administration of 30 mg Artemether Sublingual Spray 6 mg/actuation (T3) versus single oral administration of 30 mg Artemether Tablets 10 mg/tablet (T4). Mean±SD (⋅=reference, T4, □=test, T3)

FIG. 10: Plot of mean plasma Dihydroartemisinin concentration vs time with standard deviation following a single sublingual administration of 15 mg Artemether Sublingual Spray 3 mg/actuation (T1) versus single sublingual administration of 30 mg Artemether Sublingual Spray 3 mg/actuation (T2). Mean±SD (⋅=reference, T2, □=test, T1)

FIG. 11: Plot of mean plasma Dihydroartemisinin concentration vs time with standard deviation following a single sublingual administration of 30 mg Artemether Sublingual Spray 3 mg/actuation (T2) versus single sublingual administration of 30 mg Artemether Sublingual Spray 6 mg/actuation (T3). Mean±SD (⋅=reference, T3, □=test, T2)

FIG. 12: Plot of mean plasma Dihydroartemisinin concentration vs time with standard deviation following a single sublingual administration of 15 mg Artemether Sublingual Spray 3 mg/actuation (T1) versus single sublingual administration of 30 mg Artemether Sublingual Spray 6 mg/actuation (T3). Mean±SD (⋅=reference, T3, □=test, T1)

FIG. 13A: Representative chromatogram of Artemether formulation in a 20% v/v ethanol+80% v/v propellant solvent system.

FIG. 13B: Representative chromatogram of Artemether formulation in a 50% v/v ethanol+50% v/v propellant solvent system.

FIG. 13C: Representative chromatogram of Artemether formulation in an ethanol solvent system.

FIG. 13D: Representative chromatogram of Artemether formulation in a Miglyol® solvent system.

FIG. 14: Day 1 vs Day 5 Dihydroartemisinin Mean Concentration (ng/mL) vs Time for 3 mg/actuation (⋅=Day 1, □=Day 5).

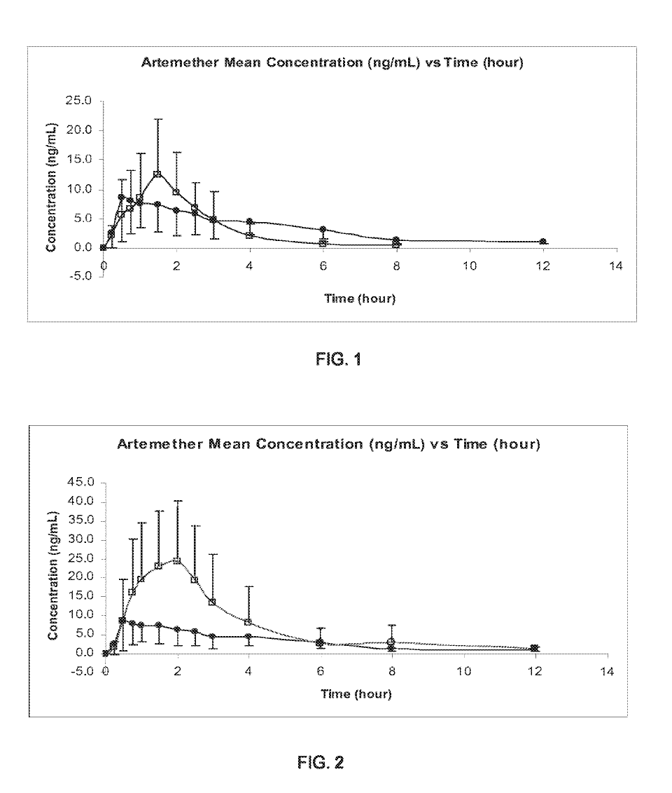

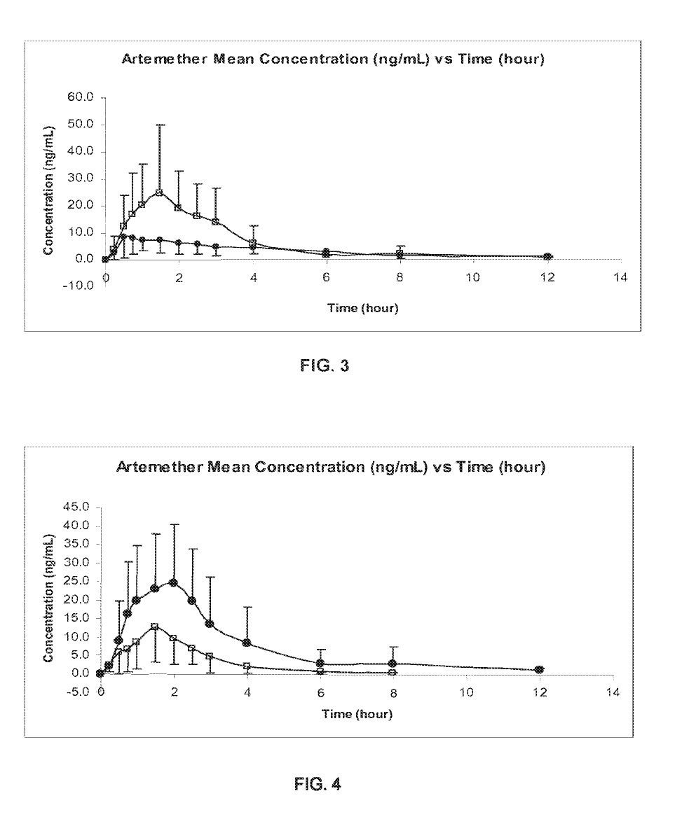

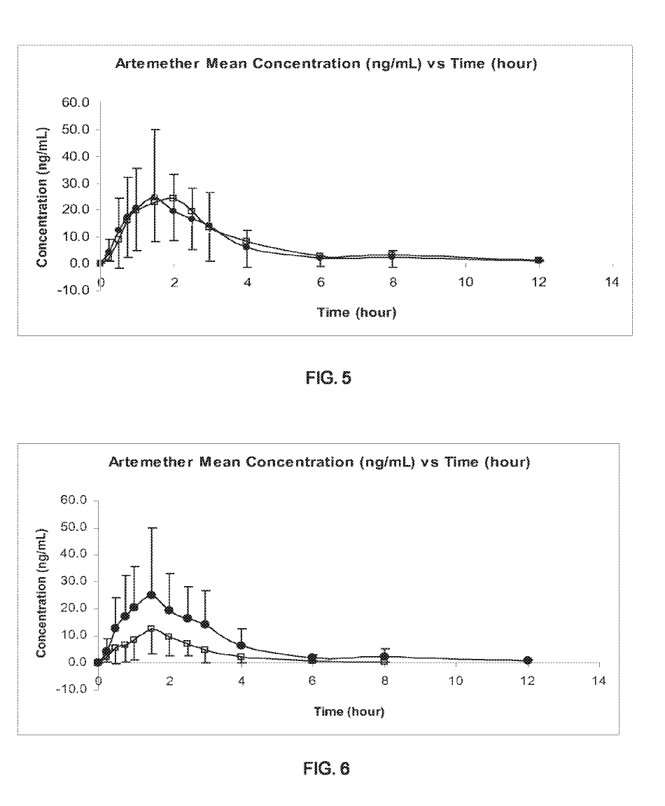

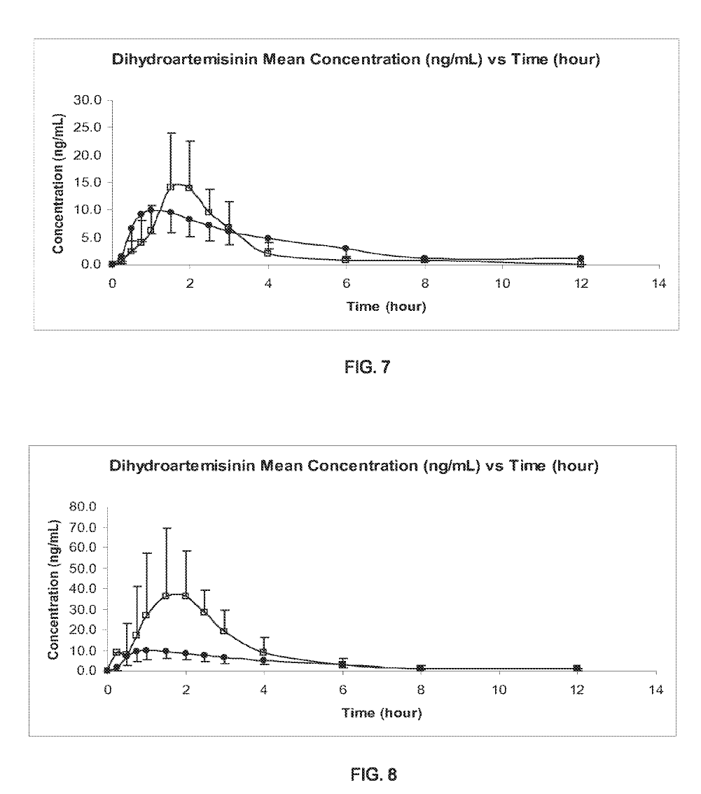

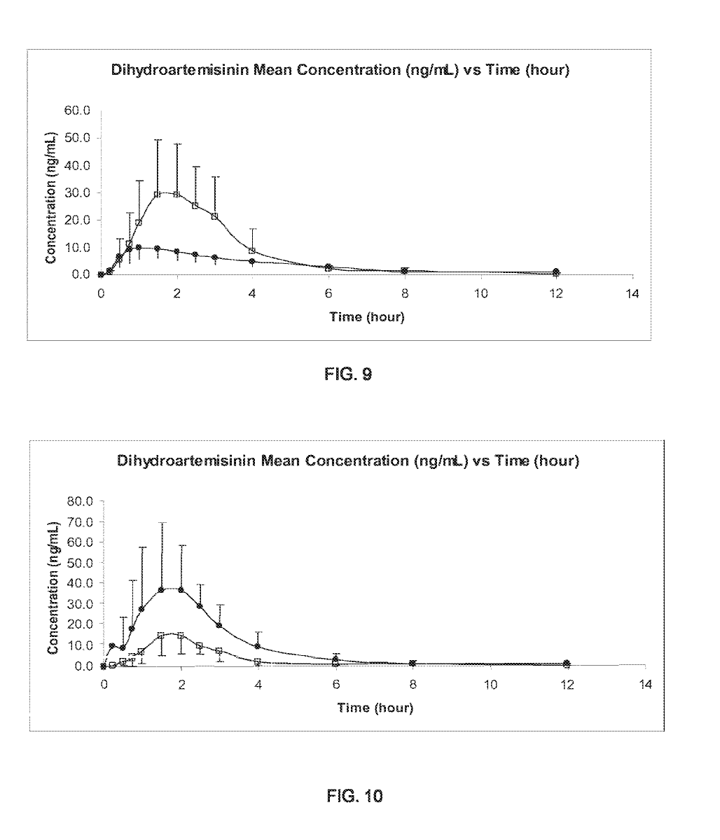

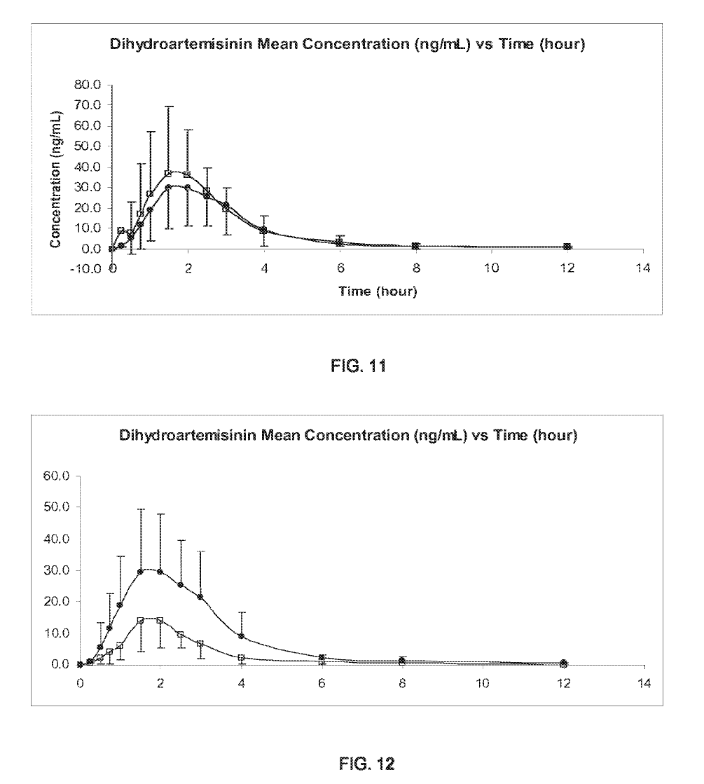

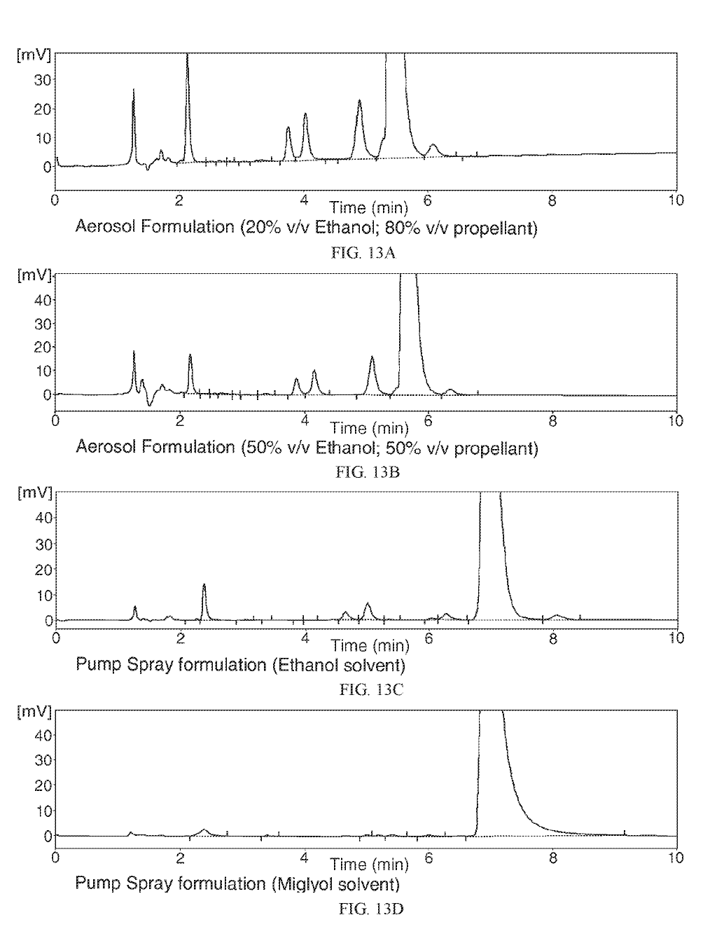

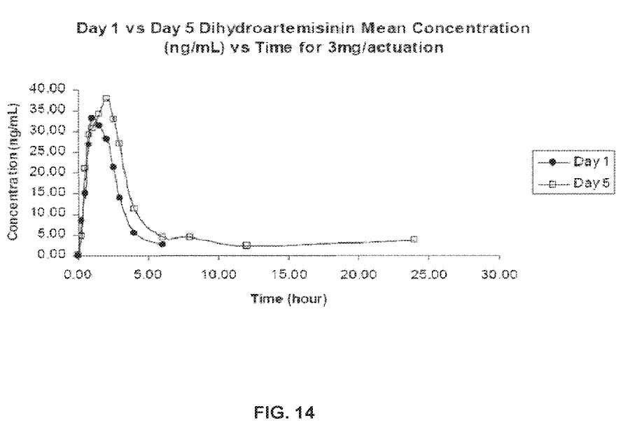

We claim:

1. A method of treating a neoplastic disease, said method comprising the administration to a patient in need thereof a therapeutically effective amount of a pharmaceutical composition by the transmucosal sublingual, buccal, or nasal route, said composition comprising: a compound capable of providing dihydroartemesinin; and a pharmaceutically-acceptable excipient selected from the group consisting of: a medium chain length triglyceride; a short chain triglyceride; an omega-3-marine triglyceride; and a fish oil, rich in omega-3-acids; said composition formulated for transmucosal sublingual, buccal, or nasal dosage.

2. The method according to claim 1, wherein said disease comprises a malignant neoplasm.

3. The method according to claim 2, wherein said disease is selected from the group consisting of: pituitary adenoma; squamous cell carcinoma; breast cancer; non-Hodgkin's Lymphoma; skin cancer; lung cancer; and non-small cell lung carcinoma.

4. The method according to claim 1, wherein said composition consists essentially of: a compound capable of providing dihydroartemesinin; and a pharmaceutically-acceptable excipient selected from the group consisting of: a medium chain length triglyceride; a short chain triglyceride; an omega-3-marine triglyceride; and a fish oil, rich in omega-3-acids; said composition formulated for transmucosal sublingual, buccal, or nasal dosage.

5. The method according to claim 1, wherein said composition consists essentially of: a compound capable of providing dihydroartemesinin; and a pharmaceutically acceptable excipient consisting essentially of: a triglyceride, which is liquid at 37° C.; and a medium chain length triglyceride; said composition formulated for transmucosal sublingual, buccal, or nasal dosage.

6. The method according to claim 1, wherein said composition is substantially free of water.

7. The method according to claim 1, wherein said composition is substantially free of ethanol.

8. The method according to claim 1, wherein said composition further comprises an essential oil selected from the group consisting of menthol, vanillin oil, orange oil, lemon oil, clove oil, peppermint oil, and spearmint oil.

9. The method according to claim 1, wherein said composition is formulated for sublingual delivery.

10. The method according to claim 1, wherein the compound capable of providing dihydroartemesinin is artemether or arteether.

11. The method according to claim 1, wherein the excipient is a medium chain length triglyceride.

12. The method according to claim 8, wherein the essential oil is menthol.

13. The method according to claim 1, wherein the compound capable of providing dihydroartemesinin is artemether, the excipient is a medium chain length triglyceride, the composition further comprises menthol, and the composition is formulated as a transmucosal sublingual dosage.

14. A method of treating a fluke infestation, said method comprising the administration to a patient in need thereof a therapeutically effective amount of a pharmaceutical composition by the transmucosal sublingual, buccal, or nasal route, said composition comprising: a compound capable of providing dihydroartemesinin; and a pharmaceutically-acceptable excipient selected from the group consisting of: a medium chain length triglyceride; a short chain triglyceride; an omega-3-marine triglyceride; and a fish oil, rich in omega-3-acids; said composition formulated for transmucosal sublingual, buccal, or nasal dosage.

15. The method according to claim 14, wherein said composition consists essentially of: a compound capable of providing dihydroartemesinin; and a pharmaceutically-acceptable excipient selected from the group consisting of: a medium chain length triglyceride; a short chain triglyceride; an omega-3-marine triglyceride; and a fish oil, rich in omega-3-acids; said composition formulated for transmucosal sublingual, buccal, or nasal dosage.

16. The method according to claim 14, wherein said composition consists essentially of: a compound capable of providing dihydroartemesinin; and a pharmaceutically acceptable excipient consisting essentially of: a triglyceride, which is liquid at 37° C.; and a medium chain length triglyceride; said composition formulated for transmucosal sublingual, buccal, or nasal dosage.

17. The method according to claim 14, wherein said composition is formulated for sublingual delivery.

18. A method of treating Lyme disease (borreliosis), said method comprising the administration to a patient in need thereof a therapeutically effective amount of a pharmaceutical composition by the transmucosal sublingual, buccal, or nasal route, said composition comprising: a compound capable of providing dihydroartemesinin; and a pharmaceutically-acceptable excipient selected from the group consisting of: a medium chain length triglyceride; a short chain triglyceride; an omega-3-marine triglyceride; and a fish oil, rich in omega-3-acids; said composition formulated for transmucosal sublingual, buccal, or nasal dosage.

19. The method according to claim 18, wherein said composition consists essentially of: a compound capable of providing dihydroartemesinin; and a pharmaceutically-acceptable excipient selected from the group consisting of: a medium chain length triglyceride; a short chain triglyceride; an omega-3-marine triglyceride; and a fish oil, rich in omega-3-acids; said composition formulated for transmucosal sublingual, buccal, or nasal dosage.

20. The method according to claim 18, wherein said composition consists essentially of: a compound capable of providing dihydroartemesinin; and a pharmaceutically acceptable excipient consisting essentially of: a triglyceride, which is liquid at 37° C.; and a medium chain length triglyceride; said composition formulated for transmucosal sublingual, buccal, or nasal dosage.
